(19) United States
(12) Patent Application Publication
(10) Pub. No.: US 2006/0107958 A1
(43) Pub. Date: May 25, 2006

(54) Adjustable sealing nasal cannula

(76) Inventor: Geoffrey P. Sleeper, Bay Village, OH (US)

Correspondence Address: PEARNE & GORDON LLP, 1801 EAST 9TH STREET, SUITE 1200, CLEVELAND, OH, 44114-3108, US

(21) Appl. No.: 11/285,235

(22) Filed: Nov. 22, 2005

Related U.S. Application Data
(60) Provisional application No. 60/629,903, filed on Nov. 22, 2004.

(51) Int. Cl. A62B 7/10; A61M 15/08

(52) U.S. Cl. 128/206.11; 128/207.18

(57) ABSTRACT

An adjustable ventilation interface for a continuous positive airway pressure system includes a nasal cannula body. The nasal cannula body includes a pair of nasal prongs that are adjustable with respect to each other. The nasal prongs are located on a top portion of the nasal cannula body to create a sealing interface between the nasal cannula body and a nose. Another sealing interface is provided by a bellows-like structure integrally molded in a portion of the nasal cannula body. The bellows portion acts as a compression spring; thereby creating an adjustable sealing between the nasal cannula body and the nose.

### CROSS REFERENCE TO RELATED APPLICATIONS

The present invention claims priority to U.S. Patent Application Ser. No. 60/629,903, filed on Nov. 22, 2004.

### FIELD OF THE INVENTION

The present invention relates generally to ventilation devices, and more particularly, to an adjustable nasal ventilation interface for a continuous positive airway pressure system.

### BACKGROUND OF THE INVENTION

Sleep apnea is a potentially life-threatening breathing disorder characterized by brief interruptions of breathing during sleep. There are two types of sleep apnea: central and obstructive. Central sleep apnea, which is less common, occurs when the brain fails to send the appropriate signals to the breathing muscles to initiate respirations. Obstructive sleep apnea occurs when air cannot flow into or out of the person's nose or mouth although efforts to breathe continue. In a given night, the number of involuntary breathing pauses or “apneic events” may be as high as 20 to 60 or more per hour. Sleep apnea can also be characterized by choking sensations. The frequent interruptions of deep, restorative sleep often leads to excessive daytime sleepiness and may be associated with an early morning headache. Early recognition and treatment of sleep apnea is important because it may be associated with irregular heartbeat, high blood pressure, heart attack, and stroke.

Various forms of positive airway pressure during sleep can be an effective form of therapy for the apnea sufferer. Ventilation can be applied in the form of continuous positive airway pressure, in which positive pressure is maintained in the airway throughout the respiratory cycle; bi-level positive airway pressure system, in which positive pressure is maintained during inspiration but reduced during expiration; and intermittent (non-continuous) positive pressure, in which pressure is applied when an episode of apnea is sensed. In such procedures, a patient wears a mask over the nose during sleep, and pressure from an air blower forces air through the nasal passages. Typically, a thin flexible tube made of an inert material transports the air. The tube terminates in an opening that can be inserted into the patient's nostrils. A pair of smaller nasal insert tubes can protrude from the tube or the tube can split at a Y-junction into two smaller tubes, each smaller nasal insert tube carrying gas to one nostril, thereby increasing the fraction of inspired oxygen.

Conventional nasal tube systems do not provide a positive seal between the nasal insert tubes and the nostrils. Most nasal ventilation systems therefore include a mask that fits over the nose and is intended to provide a space of oxygen-enriched air for inhalation into the lungs for respiration. Such systems frequently suffer from air leaking out around the mask, creating an inability to assure ventilation in many patients. Additionally, most systems are usually very position dependent, whereby if the mask is moved slightly with respect to the facial contour or with respect to the nose, air leakage occurs. With such systems, the mask can become uncomfortable when not in position, thus requiring the patient to remain rather still in order to alleviate the discomfort and to maintain oxygen inspiration.

### SUMMARY OF THE INVENTION

The following presents a simplified summary of the invention in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In accordance with an aspect of the present invention, a ventilation interface is provided. The ventilation interface includes a nasal cannula body, which comprises: a pair of nasal prongs located on a top portion of the nasal cannula body; and a first bellows-like structure positioned between the nasal prongs, the first bellows-like structure being configured to provide adjustability in a center-to-center distance between the nasal prongs.

In accordance with another aspect of the present invention, a ventilation interface is provided. The ventilation interface includes: a nasal cannula body; a pair of nasal prongs located on a top portion of the nasal cannula body; a first bellows-like structure positioned between the pair of nasal prongs; and a second bellows-like structure positioned between a top surface of the nasal cannula body and a bottom surface of the nasal cannula body, wherein the first bellows-like structure is configured to expand and contract in a direction that is substantially transverse to the direction in which the second bellows-like structure is configured to expand and contract.

In accordance with yet another aspect of the present invention, a ventilation interface is provided. The ventilation interface includes: a nasal cannula body; and a pair of nasal prongs located on a top portion of the nasal cannula body, the nasal prongs comprising a vertical corrugations having a whorled configuration such that pressure inside the nasal prongs can cause the nasal prongs to expand with a slight twisting motion.

In accordance with yet another aspect of the present invention, a ventilation interface is provided. The ventilation interface includes: means for adjusting a center-to-center distance between two nasal prongs projecting from a top portion of a nasal cannula body; means for creating a first sealing interface between a top surface of the nasal cannula body and a bottom surface of a patient's nose; and means for creating a second sealing interface between an outer surface of the nasal prongs and an inner surface of the patient's nose.

The following description and the annexed drawings set forth in detail certain illustrative aspects of the invention. These aspects are indicative, however, of but a few of the various ways in which the principles of the invention may be employed and the present invention is intended to include all such aspects and their equivalents. Other objects, advantages and novel features of the invention will become apparent from the following detailed description of the invention when considered in conjunction with the drawings.

### DETAILED DESCRIPTION OF THE INVENTION

The present invention provides a nasal ventilation interface having at least two sealing interfaces. The present invention will now be described with reference to the drawings, wherein like reference numerals are used to refer to like elements throughout. It is to be appreciated that the various drawings are not necessarily drawn to scale from one figure to another nor inside a given figure, and in particular that the size of the components are arbitrarily drawn for facilitating the reading of the drawings. In the following description, for purposes of explanation, numerous specific details are set forth in order to provide a thorough understanding of the present invention. It may be evident, however, that the present invention may be practiced without these specific details.

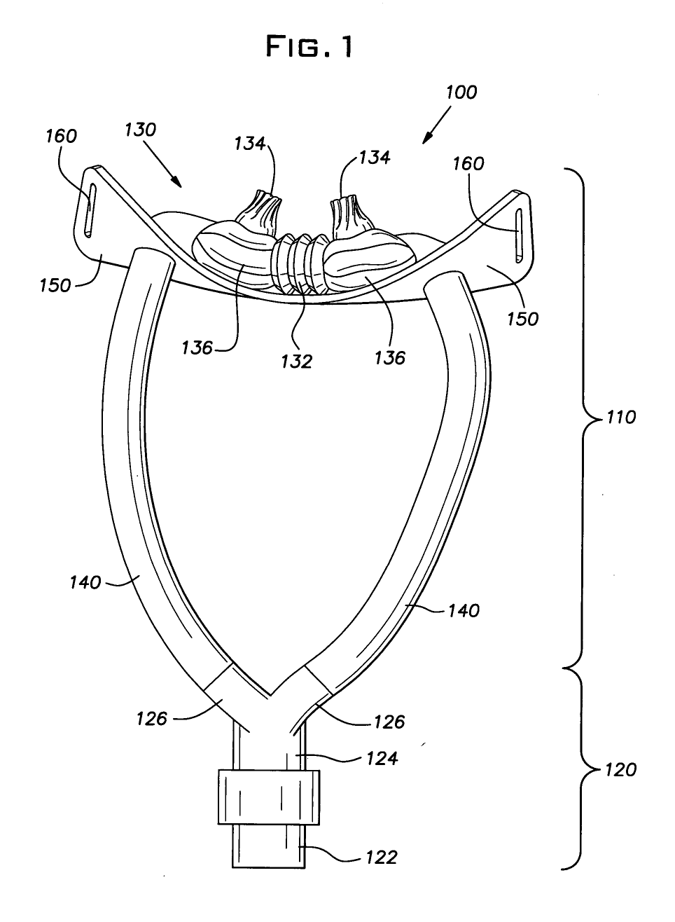

Turning initially to FIG. 1, an example of a nasal ventilation interface 100 in accordance with a first aspect of the present invention is illustrated. The nasal interface 100 comprises a base portion 110 and a swivel component 120. The base portion 110 includes a nasal cannula body 130 materially integral with two supply tubes 140. The base portion 110 is manufactured from one or more inert materials, such as polyurethane, silicone, or the like. The supply tubes 140 are employed to deliver air pressure from a ventilation device (not shown) to a patient via the nasal cannula body 130. In particular, the ventilation device forces a gas, such as air, through the supply tubes 140 and can be provided by a continuous positive airway pressure machine, a bi-level positive airway pressure machine, an intermittent (non-continuous) positive pressure machine, or any other suitable machine to deliver air to the patient.

For sleep apnea therapy, the ventilation device will usually supply room air at a pressure of between five and fifteen centimeters of water. The room air may be supplemented with oxygen if desired by splicing an oxygen supply line into the supply hose or using a triple port connector. It is normally unnecessary to humidify or add moisture to the air supplied by the ventilation device in using the nasal interface of the present invention, as the nasal interface is designed to avoid stripping moisture from the nares. Thus, moisture does not have to be added to relieve patient discomfort from drying or burning sensations in the nasal airways.

Each of the supply tubes 140 includes an end portion, which is coupled to the swivel component 120 to facilitate easy manipulation of the supply tubes 140 for patient comfort. The swivel component 120 comprises a substantially cylindrical element 122 for coupling with a tube of the ventilation device and a coupling member 124 having two tubular engaging portions 126 projecting therefrom. The two tubular engaging portions 126 are utilized for coupling with end portions of the supply tubes 140 of the ventilation interface 100. The cylindrical element 122 and the coupling member 124 are operable to swivel with respect to each other. For instance, the cylindrical element 122 and the coupling member 124 can swivel about each other by 360°. It is to be appreciated that any suitable structure contemplated for swiveling the ventilation interface 100 with the tube of the ventilation device can be utilized.

The nasal interface 100 also includes headgear strap flanges 150, which are coupled to the base portion 110, to facilitate utilization of headgear straps (not shown). It is to be appreciated that the headgear strap flanges 150 can be materially integral with the nasal cannula body and/or the supply tubes 140 or the headgear strap flanges 150 can be separate components adapted to couple with at least one of the nasal cannula body 130 and the supply tubes 140. Each of the headgear strap flanges 150 includes at least one aperture 160 for receiving a portion of the headgear straps therethrough. When nasal prongs of the nasal cannula body 130 are inserted into nares of the patient, the headgear strap fastens around the patient's head and applies backward pressure to the nasal cannula body 130. A first sealing interface is thus created via the headgear strap securing the nasal interface 100 against the patient's mustache region. In addition to this backward pressure, the flanges 150 are positioned in such a way that the headgear strap applies an angular, upward pressure (e.g., approximately a 45-degree angle) to a bellows portion of the nasal cannula body 130, which will be described in further detail below. This angular, upward pressure creates a second sealing interface between the nasal cannula body 130 and the patient's nose.

The supply tubes 140 can be shaped to extend along a base of the nasal cannula body 130 and bend downward near the headgear strap flanges 150. As a result, the headgear straps support weight and torque produced by the supply tubes 140, thereby decreasing the chance of the supply tubes 140 disturbing a sealing means and potentially breaking a seal between the ventilation interface 100 and the patient. Alternatively or additionally, the supply tubes 140 can be looped over the patient's ears.

The nasal cannula body 130 of the ventilation interface 100 will now be described in greater detail. The nasal cannula body 130 provides adjustability in several areas. A first bellows-like structure 132 is positioned between a pair of nasal prongs 134 and is configured to compress and expand. The compression and expansion of the first bellows-like structure 132 provides adjustability in a center-to-center distance between the nasal prongs 134, which in turn provides greater comfort to the patient. The first bellows-like structure 132 can be integrally molded into the nasal cannula body 130. Alternatively, the first bellows-like structure 132 can be a separate component employed to join two separate nasal prong components. Each nasal prong is a separate piece that slides back and forth via the first bellows-like structure. It is to be appreciated that any bellows-like configuration provided between the nasal prongs to provide adjustability of the nasal prongs is within the scope of the present invention.

Optionally or additionally, the nasal cannula body 130 includes a second bellows-like structure 136 that can be positioned between a top surface of the nasal cannula body 130 and a bottom surface of the nasal cannula body 130. The second bellows-like structure 136 can be integrally molded in the nasal cannula body 130. The second bellows-like structure is configured to facilitate an improved sealing interface between the nasal cannula body 130 and the patient's nose. More specifically, the improved sealing interface is created between a top surface of the nasal cannula body 130 and a bottom, triangular shaped area of the nose. The second bellows-like structure 136 acts in a manner similar to a compression spring to apply a gentle upward pressure to the nose thereby holding the sealing surfaces (e.g., the top surface of the nasal cannula body 130 and the bottom area of the nose) in sealing engagement with one another. The second bellows-like structure 136 is adjustable in length between a contracted state and an expanded state.

The first and second bellows-like structures 132 and 136 can include the bellows feature substantially around the periphery of each structure for increased flexibility or can only be provided around a portion of the structure for increased rigidity. Further, first bellows-like structure 132 can be configured to expand and contract in a direction that is substantially transverse to the direction in which the second bellows-like structure 136 is configured to expand and contract.

The nasal prongs 134 extending from the top portion of the nasal cannula body create another sealing interface between an outer surface area of the nasal prongs 134 and an inner surface area of the patient's nares. FIG. 1 illustrates one example of a nasal prong configuration that can be employed with the present invention. The nasal prongs 134 feature a series of vertical corrugations, which allow the nasal prongs 134 to expand in the nares and seal a very wide range of anatomical sizes and shapes. The vertical corrugations can have a whorled configuration such that pressure inside the nasal prongs 134 can cause the nasal prongs 134 to expand with a slight twisting motion; thereby providing a wide range of expansion. The walls of the nasal prongs 134 are of a thickness such that they are able to inflate under pressure. For example, the nasal prongs 134 can be easily and comfortably inserted into a nose of a patient in a compressed state, as illustrated in FIG. 1. Then, when a gas flows through the ventilation interface via a CPAP machine, for example, the nasal prongs 134 can inflate to create an air tight sealing surface between the outer surface of the nasal prongs 134 and the nares of the patient. This allows the prongs 134 to expand in the nares and seal a wide range of anatomical sizes and shapes. The nasal prongs 134 can assume a barrel-shaped structure when inflated to provide a large, even sealing surface in the nares. However, it is to be appreciated that the nasal prongs 134 can assume any suitable shape when inflated to provide maximum sealing between the prongs 134 and the nares.

It is to be appreciated that the nasal prong configuration having the whorled vertical corrugations can be employed in any type of nasal cannula body and is not limited to nasal cannula body 130, as shown and described in FIG. 1. For instance, the nasal prongs having whorled vertical corrugations can be employed in a nasal cannula body having only one bellows-like structure, more than one bellows-like structure, or no bellows-like structure and is contemplated as falling within the scope of the present invention.

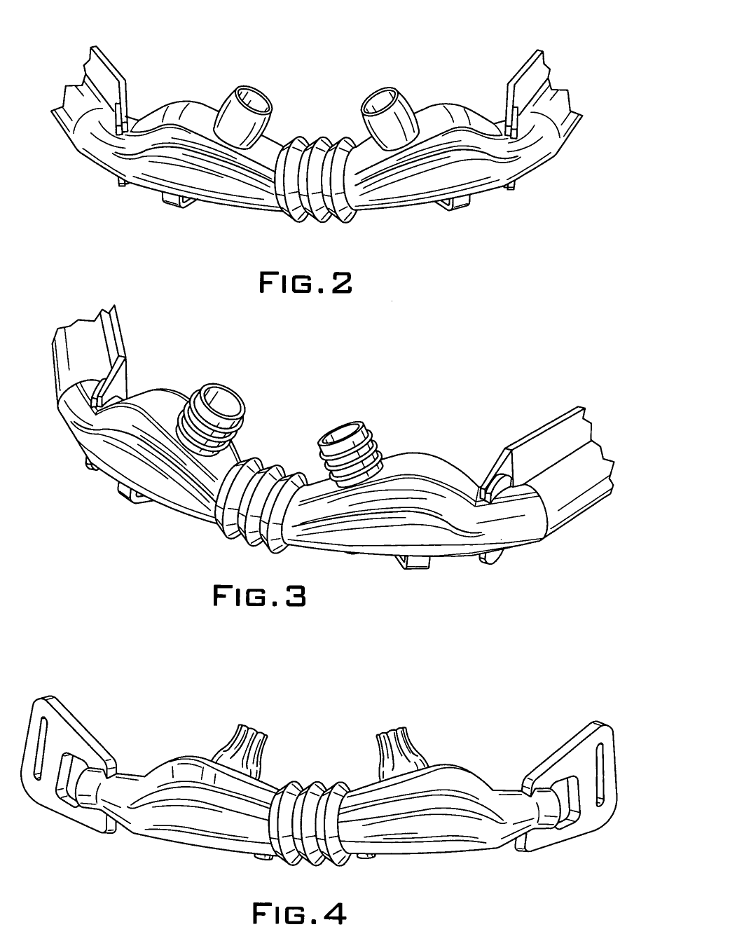

Alternatively, the nasal cannula body 130 can include two substantially barrel-shaped nasal prongs 210, as illustrated in FIG. 2. The nasal prongs 210 operably create a sealing interface between the nasal prongs 210 and the patient's nares via the barrel-shaped structure. The ‘barrel shape’ is defined by a diameter of a central portion of the nasal prongs 210 being greater than diameters at end portions of the nasal prongs 210. Employing such a barrel shape structure creates a large, even sealing surface when inserted into the patient's nares. For instance, when inserted into the nares of the patient, the barrel shape of each of the prongs 210 is compressed in a radial direction such that a substantially uniform pressure is applied across the outer surface of each of the prongs 210 and against an inner surface of a respective naris, thus forming a substantially airtight seal between the prong 210 and the naris over a large surface area. The barrel shaped nasal prongs 210 can be employed with a nasal cannula body having one or more vertical bellows-like structure provided between the nasal prongs 210 and/or one or more horizontal bellows-like structure provided within the nasal cannula body.

As another alternative nasal prong configuration, FIG. 3 illustrates a pair of nasal prongs 410 comprising a substantially straight-shaped, hollow body having two or more rings 420 provided around an outer surface thereof. For example, the nasal prongs 410 can include three rings. A sealing interface is created between an outer surface of the rings 420 and an inner surface of a patient's nares when the nasal prongs 410 are inserted into a nose of a patient. It is to be appreciated that the rings 420 can also be used in combination with the barrel-shaped nasal prongs 210 described with respect to FIG. 2.

FIG. 4 illustrates another alternative nasal prong configuration that can be employed with any of the nasal ventilation interfaces disclosed herein. The nasal prongs 1310 have thin, ribbed walls, which are adapted to inflate under pressure. For example, the nasal prongs 1310 can be easily and comfortably inserted into a nose of a patient in a compressed state, as illustrated in FIG. 4. Then, when a gas flows through the ventilation interface via a CPAP machine, for example, the nasal prongs 1310 can inflate to create an air tight sealing surface between the outer surface of the nasal prongs 1310 and the nares of the patient. The nasal prongs 1310 can assume a barrel-shaped structure when inflated to provide a large, even sealing surface in the nares. However, it is to be appreciated that the nasal prongs 1310 can assume any suitable shape when inflated to provide maximum sealing between the prongs 1310 and the nares.

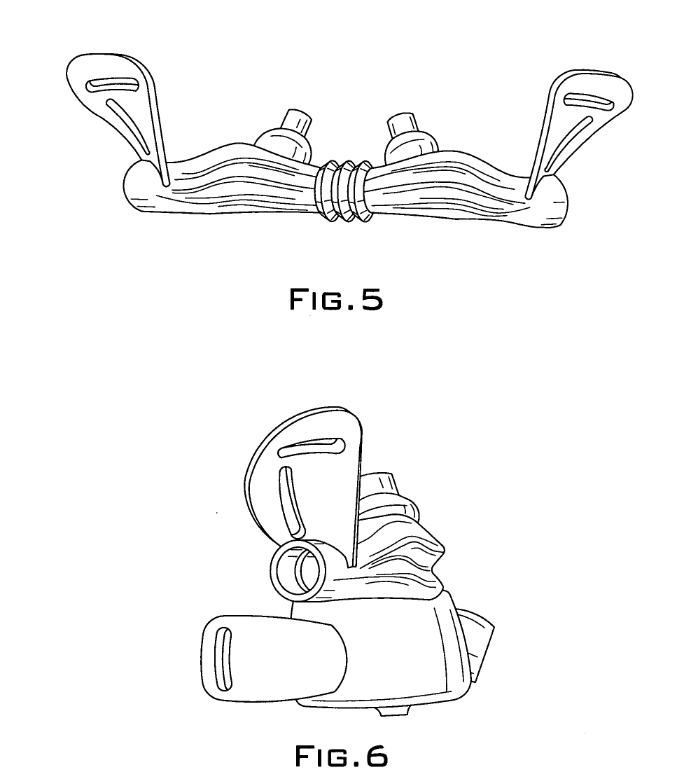

Another alternative nasal prong configuration is illustrated in FIG. 5. The nasal prongs 2030 include a bulbous base portion that tapers into a substantially straight top portion. The nasal prongs 2030 are inserted into the nares of the patient such that the bulbous base portion of the nasal prongs 2030 creates a substantially airtight seal between an outer surface area of the base portion and an inner surface area of the nares.

Although not shown, the nasal ventilation interface can also include one or more swivel elbows to provide an airtight coupling between the nasal cannula body and the supply tubes, as well as, to provide an additional swivel feature to the nasal ventilation interface. The swivel elbows swivel about an axis parallel to a central axis of inlet ports of the nasal cannula body; thereby, allowing the supply tubes to swivel 360° about the nasal cannula body. Thus, the patient can wear the nasal ventilation interface with the supply tubes down towards their chest or above their head. Further, the swivel elbows allow the nasal cannula body to self-adjust to a correct angle for nasal prong insertion in both the downward and over the head positions.

The swivel elbows can be manufactured from a rigid plastic material, or any other suitable material, and include an elbow component, a swivel connector, and a locking collar. The swivel connector fits over an end portion of the elbow component. The locking collar snaps over a portion of the swivel connector such that at least one small protrusion (not shown) on the locking collar projects through a corresponding aperture on the swivel connector to make contact with the elbow component, thereby locking the three components together. The swivel connector and the locking collar are then operable to rotate about the end portion of the elbow component. It is to be appreciated that any suitable size and shape swivel component can be employed to couple at least one supply tube to the nasal cannula body and is contemplated as falling within the scope of the present invention.

FIG. 6 illustrates another example of a nasal ventilation device. The nasal ventilation device is a hybrid of a nasal cannula body portion and a face mask portion. The nasal cannula body portion includes a pair of nasal prongs for insertion into a patient's nares. The nasal prongs can include any of the plurality of configurations disclosed herein. At least one inlet is included on the nasal cannula body portion for receiving the gas from the ventilation device (not shown).

The nasal cannula body portion further includes at least one bellows-like structure formed within the nasal cannula body portion to facilitate adjustability of the nasal ventilation interface. Headgear strap flanges can also be integrally formed with the nasal cannula body portion to facilitate yet another sealing interface between the nasal cannula body portion and the patient. The headgear strap flanges each include at least one aperture, and in this example, each of the headgear strap flanges includes two apertures. The apertures receive headgear straps, which are then fastened around the patient's head. The position of the headgear strap flanges, as well as the positions of the apertures, pull the nasal cannula body portion backwards and upwards towards the patient's face to create a sealing interface between a back portion of the nasal cannula body and the patient's mustache region.

The face mask portion of the ventilation device includes an elastomeric material and is shaped so as to fit the contours of a patient's face around a mouth area of the patient. The face mask portion also includes headgear strap flanges formed integrally with the mask to facilitate sealing of the mask against the patient's face. The headgear strap flanges each include at least one aperture for receiving headgear straps. The face mask portion further includes at least one bleeder port and an anti-asphycsia valve.

Due to the different sealing means of a nasal ventilation interface, as described with respect to the plurality of embodiments described herein, an adequate seal is provided with minimal pressure concentration being applied to the patient's nose and face; thereby, mitigating mucosal irritation. Accordingly, effectiveness as well as comfort of the nasal ventilation interface is achieved. Further, due to the adjustability of the ventilation interface as described herein, the ventilation interface can adapt to fit a large patient population with a single device, thereby substantially mitigating the need for multiple ventilation interface sizes.

Although a detailed description of a preferred embodiment of this invention has been shown and described hereinabove, it will be understood that various modifications and rearrangements of the parts and their respective features may be resorted to without departing from the scope of the invention as disclosed herein.

What is claimed is:

1. A ventilation interface comprising:
a nasal cannula body, the nasal cannula body comprising:
a pair of nasal prongs located on a top portion of the nasal cannula body; and
a first bellows-like structure positioned between the nasal prongs, the first bellows-like structure being configured to provide adjustability in a center-to-center distance between the nasal prongs.

2. The ventilation interface of claim 1, wherein the nasal prongs are substantially barrel-shaped to create a large sealing surface when inserted into the nose.

3. The ventilation interface of claim 1, wherein the nasal prongs include two or more rings provided thereon to create a sealing surface between an outer surface of the rings and an inner surface of a patient's nares.

4. The ventilation interface of claim 1, wherein the nasal prongs include a thin, ribbed wall that inflates under pressure.

5. The ventilation interface of claim 4, wherein the nasal prongs are substantially barrel-shaped when inflated under pressure.

6. The ventilation interface of claim 1, wherein the nasal prongs include a bulbous-shaped base portion.

7. The ventilation interface of claim 6, wherein the bulbous-shaped base portion tapers into a straight-shaped end portion.

8. The ventilation interface of claim 1, wherein the first bellows-like structure is materially integral with the nasal cannula body.

9. The ventilation interface of claim 1, wherein the first bellows-like structure is a separate component from each of the nasal prongs.

10. The ventilation interface of claim 1, further comprising a second bellows-like structure positioned between a top surface and a bottom surface of the nasal cannula body, the second bellows-like structure being configured to create a sealing interface between the top surface of the nasal cannula body and a bottom surface of a patient's nose.

11. The ventilation interface of claim 10, wherein the second bellows-like structure is materially integral with the nasal cannula body.

12. The ventilation interface of claim 1, further comprising a pair of supply tubes for delivering a gas to a patient via the nasal cannula body.

13. The ventilation interface of claim 12, wherein the supply tubes are formed integrally with the nasal cannula body.

14. The ventilation interface of claim 12, wherein the supply tubes are coupled to the nasal cannula body via at least one swivel component.

15. The ventilation interface of claim 1, further comprising a pair of flanges for securing a headgear strap thereto.

16. The ventilation interface of claim 15, wherein the flanges are formed integrally with the nasal cannula body.

17. The ventilation interface of claim 15, wherein a first flange is formed integrally with a first inlet port of the nasal cannula body and a second flange is formed integrally with a second inlet port of the nasal cannula body.

18. The ventilation interface of claim 15, wherein the flanges are positioned at an angle of about 45-degrees with respect to a central axis of an inlet port formed integrally with the nasal cannula body.

19. The ventilation interface of claim 1, wherein a main portion of the nasal cannula body is shaped to conform to a mustache area of a patient's face.

20. A ventilation interface comprising:
a nasal cannula body;
a pair of nasal prongs located on a top portion of the nasal cannula body;
a first bellows-like structure positioned between the pair of nasal prongs; and
a second bellows-like structure positioned between a top surface of the nasal cannula body and a bottom surface of the nasal cannula body,
wherein the first bellows-like structure is configured to expand and contract in a direction that is substantially transverse to the direction in which the second bellows-like structure is configured to expand and contract.

21. The ventilation interface of claim 20, wherein each of the first and second bellows-like structures are materially integral with the nasal cannula body.

22. The ventilation interface of claim 20 wherein the nasal prongs comprise a thin, ribbed wall that inflates under pressure to create the sealing surface.

23. The ventilation interface of claim 22, wherein the thin, ribbed wall is substantially vertically corrugated.

24. The ventilation interface of claim 22, wherein the thin, ribbed wall comprises a whorled configuration such that pressure inside the nasal prongs can cause the nasal prongs to expand with a slight twisting motion.

25. A ventilation interface comprising:
a nasal cannula body; and
a pair of nasal prongs located on a top portion of the nasal cannula body, the nasal prongs comprising a vertical corrugations having a whorled configuration such that pressure inside the nasal prongs can cause the nasal prongs to expand with a slight twisting motion.

26. The ventilation interface of claim 25, further comprising a first bellows-like structure positioned between the nasal prongs, the first bellows-like structure being configured to expand and contract to provide adjustability of a center-to-center distance between the nasal prongs.

27. The ventilation interface of claim 26, further comprising a second bellows-like structure positioned between a top surface of the nasal cannula body and a bottom surface of the nasal cannula body, the second bellows-like structure being configured to provide a sealing interface between the top surface of the nasal cannula body and a bottom surface of a patient's nose.

28. A ventilation interface comprising:
means for adjusting a center-to-center distance between two nasal prongs projecting from a top portion of a nasal cannula body;
means for creating a first sealing interface between a top surface of the nasal cannula body and a bottom surface of a patient's nose; and
means for creating a second sealing interface between an outer surface of the nasal prongs and an inner surface of the patient's nose.
